(19) United States
(12) Patent Application Publication
(10) Pub. No.: US 2025/0169952 A1
(43) Pub. Date: May 29, 2025

(54) DEVICES AND METHODS FOR TRICUSPID VALVE ANNULOPLASTY

(71) Applicant: The Board of Trustees of the Leland Stanford Junior University, Stanford, CA (US)

(72) Inventors: Y. Joseph Woo, Stanford, CA (US); Pearly Pandya, Stanford, CA (US); Matthew Park, Piedmont, CA (US); Yuanjia Zhu, Stanford, CA (US)

(21) Appl. No.: 19/036,943

(22) Filed: Jan. 24, 2025

Related U.S. Application Data
(63) Continuation of application No. PCT/US2023/028898, filed on Jul. 27, 2023.
(60) Provisional application No. 63/392,825, filed on Jul. 27, 2022.

(51) Int. Cl. A61F 2/24

(52) U.S. Cl. CPC A61F 2/2448

(57) ABSTRACT

Annuloplasty devices are provided for implantation around a tricuspid valve to avoid placement over a septal leaflet of the valve and/or avoid suture placement adjacent the septal leaflet. In one example, the device includes a “C” shaped body that includes spaced apart ends to provide a gap to avoid placement over the septal leaflet. Alternatively, a flexible portion may be connected to the ends of the “C” shaped body for placement over the septal region. In another example, the device includes an enclosed band including a contoured bridge configured to sit over the septal leaflet, e.g., to avoid unnecessary leaflet abrasion and/or conform to the septal wall and aorta.

### RELATED APPLICATION DATA

The present application is a continuation of co-pending International Application no. PCT/US2023/028898, filed Jul. 27, 2023, which claims benefit of U.S. provisional application claims Ser. No. 63/392,825, filed Jul. 27, 2022, the entire disclosures of which are expressly incorporated by reference herein.

### TECHNICAL FIELD

The present application relates to medical devices and, more particularly, to annuloplasty devices and to methods for making and using such devices.

### STATEMENT REGARDING FEDERALLY SPONSORED RESEARCH AND DEVELOPMENT

None.

### BACKGROUND

Annuloplasty rings are designed and implanted to restore diseased/regurgitant valves, e.g., tricuspid valves, to their normal size and shape. If left untreated, tricuspid valve regurgitation can lead to debilitating symptoms including congestive heart failure and irreversible heart damage. Ring annuloplasty has been associated with significantly greater durability of tricuspid repair compared with suture annuloplasty. With tricuspid ring annuloplasty, the valve annulus is remodeled or permanently fixed by suturing an incomplete ring, and suture placement is avoided in regions of conductive tissue and the bundle of His to minimize risk of conduction block. However, the rate of permanent pacemaker associated with this surgery has been increasing in recent years. Avoiding conduction block and the need for permanent pacemaker is crucial, since transvalvular pacemaker leads are associated with much greater recurrence rates of tricuspid regurgitation.

Therefore, annuloplasty devices that conform to anatomical structures and/or do not interfere with conduction tissues would be useful.

### SUMMARY

The present application is directed to medical devices and, more particularly, to annuloplasty devices and to methods for making and using such devices.

For example, provided herein are novel annuloplasty devices for implantation around a tricuspid valve annulus, e.g., that conform around the septal region of the tricuspid valve. Most commercially available tricuspid bands have a portion that crosses the septal leaflet (i.e., have a much smaller gap between the two ends of the band), so suturing in these regions introduces the risk of damaging conductive tissue and the bundle of His, which is located adjacent the septal leaflet.

Several exemplary configurations are described herein. For example, a first exemplary device includes a “C” shaped band that includes ends that are spaced apart to avoid placement over the septal leaflet of a tricuspid valve (Configuration A). A second exemplary device includes an enclosed band that includes a “C” shaped body that includes spaced apart ends, and a flexible portion (e.g., including flexible polymer and fabric) connecting the ends of the “C” shaped body for placement over the septal region (Configuration B), which may help retain the position of the two ends of the band, but not require suture placement.

A third exemplary device includes an enclosed band including a contoured bridge configured to sit over the septal leaflet of a tricuspid valve in the right atrium. The bridge may allow for retention of the two ends of the band. In addition, a height of the bridge may be configured to avoid unnecessary leaflet abrasion or hemolysis, and/or an inward angle of the bridge may be configured to conform to the septal wall and aorta. These three configurations may be provided separately or as a modular unit to a surgeon or other user.

Each of these configurations may include a flexible portion adjacent one end, e.g., configured to positioned near the aorta. In one example, the materials used to make the devices may include metal wire, biocompatible polymers, and fabric and the devices may be formed by one or more of molding, casting, 3D printing, machining, and the like.

In accordance with one example, an annuloplasty device is provided for implantation around a tricuspid valve of a heart that includes a body including a central region, first and second end regions extending from opposite ends of the central region, and a bridge extending between the first and second end regions opposite the central region to define an enclosed ring configured to avoid interference with a septal leaflet of the tricuspid valve.

In accordance with another example, an annuloplasty device is provided for implantation around a tricuspid valve of a heart that includes a body including a curved central region and curved first and second end regions extending from opposite ends of the central region lying generally within a plane, and a bridge extending between the first and second end regions opposite the central region to define an enclosed ring, the bridge extending out of the plane between the first and second end regions to avoid interference with a septal leaflet of the tricuspid valve.

In accordance with still another example, an annuloplasty device is provided for implantation around a tricuspid valve of a heart that includes a body including a central region and first and second end regions extending from opposite ends of the central region such that tips of the end regions are spaced apart from one another, thereby generally defining a “C” shape.

In accordance with another example, a method is provided for performing annuloplasty that includes providing an annuloplasty device comprising a body including a central region and first and second end regions extending from opposite ends of the central region such that tips of the end regions are spaced apart from one another, thereby generally defining a “C” shape; positioning the device around a tricuspid valve annulus such that the tips of the first and second end regions are located on either side of a septal leaflet of the tricuspid valve with a gap between the tips; and securing the device to tissue adjacent the annulus. Optionally, a distance of the gap between the tips of the first and second ends is sized to avoid placement of the tips over the septal leaflet when the body is secured around anterior and posterior leaflets of the tricuspid valve.

In accordance with yet another example, a method is provided for performing annuloplasty that includes providing an annuloplasty device comprising a body including a central region, first and second end regions extending from opposite ends of the central region, and a bridge extending between the first and second end regions opposite the central region to define an enclosed ring; positioning the device around a tricuspid valve annulus such that the first and second end regions are located on either side of a septal leaflet of the tricuspid valve and the bridge avoids interference with the septal leaflet; and securing the device to tissue adjacent the annulus. To secure the device, one or more sutures or other fasteners may be introduced through fabric covering the device into the tissue adjacent the annulus, e.g., with no sutures or fasteners delivered through the bridge to avoid damaging conductive tissue or the bundle of His.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIGS. 1A and 1B show first and second examples of annuloplasty devices that may be implanted around a tricuspid valve within a subject's heart.

FIGS. 2A-2E are various views of another example of an annuloplasty ring that may be implanted around a tricuspid valve within a subject's heart.

FIGS. 3A and 3B show the annuloplasty ring of FIGS. 2A-2E implanted around a tricuspid valve.

FIG. 4 shows an apparatus for testing annuloplasty devices such as the exemplary annuloplasty ring shown, e.g., to identify resistive forces of the devices.

FIG. 5 is a graph showing exemplary resistive forces that were measured from tests using the apparatus shown in FIG. 4.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

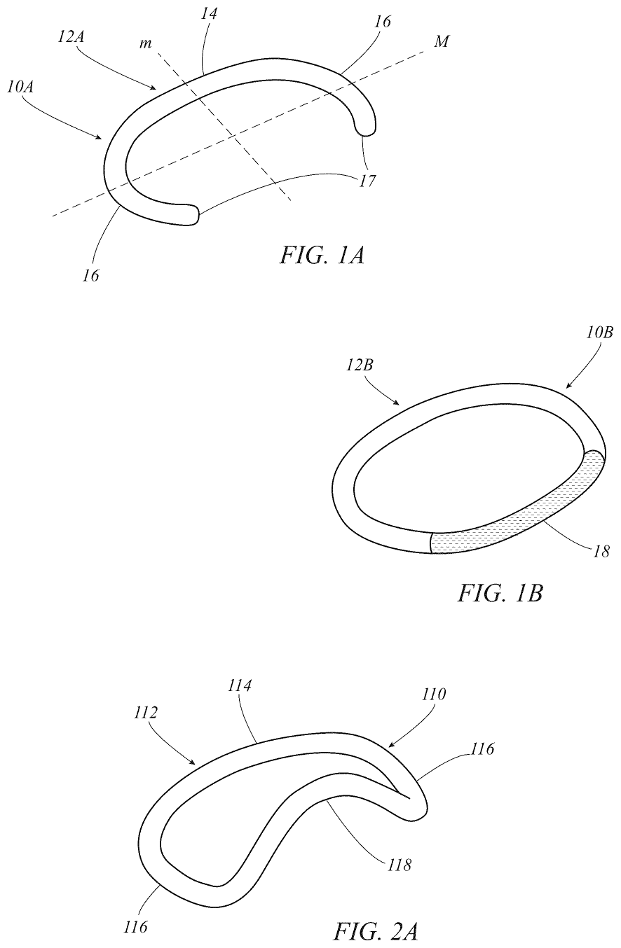

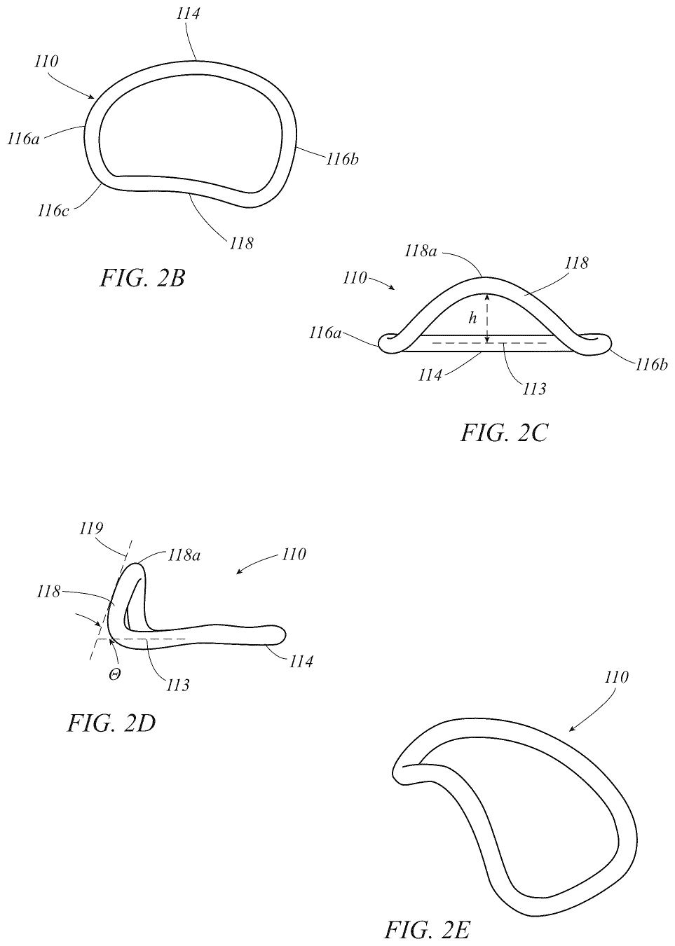

Turning to the drawings, FIGS. 1A, 1B, and 2A-2E show examples of an annuloplasty device that may be implanted around a tricuspid valve within a subject's heart.

For example, FIG. 1A shows an annuloplasty device 10A that includes a “C” shaped body 12 including a central or intermediate region 14 and first and second end regions 16 extending from opposite ends of the central region 14 such that tips 17 of the end regions 16 are spaced apart from one another, e.g., defining a “C” shape lying generally within a plane. The body 12 is sized to located around a tricuspid valve annulus with the central region 14 opposite the septal leaflet and the tips 17 of the first and second end regions 16 located on either side of a septal leaflet of the tricuspid valve with a gap between the tips 17. For example, the length of the gap between the tips of the first and second end regions 16 is sized to avoid placement of the tips 17 over the septal leaflet when the body 12 is secured around anterior and posterior leaflets of the tricuspid valve. Thus, sutures used to secure the device 10A may be directed through tissue surrounding the annulus, while avoiding suturing in regions adjacent the septal leaflet, which may otherwise risk damaging conductive tissue or the bundle of His.

As shown, the body 12 may have an oblong shape, e.g., with the central region 14 having a larger radius of curvature than the end regions 16, such that a length of the body 12 between the end regions 16 along a major axis “M” is greater than a width along a minor axis “m” orthogonal to the major axis M. The shape of the end regions 16 may be substantially the same, e.g., symmetrically opposite one another but having the same length and radius of curvature. Alternatively, the end regions may be asymmetric, if desired.

The body 12 may be formed from biocompatible materials, e.g., polymeric material formed around a core wire to define an outer surface of the body. Fabric, e.g., Dacron and the like (not shown), may be provided around the body to cover the outer surface. The resulting device may be substantially rigid or semi-rigid, e.g., to support the annulus of the tricuspid valve. Alternatively, the material may be malleable, e.g., such that the shape may be modified by a surgeon to conform to the anatomy of an individual patient before implantation. In this alternative, the force necessary to manipulate the shape should be greater than the maximum forces the device may experience after implantation.

For example, the body 12 may include a substantially rigid core wire (not shown), e.g., formed from metal such as stainless steel, titanium, Elgiloy, Nitinol, plastic, or component materials that extends through the central region 14 and end regions 16. A rigid, semi-rigid, or flexible outer material, e.g., elastomeric material such as silicone, a polymer such as urethane methacrylate, and the like, may be provided around the core wire to provide some flexibility and/or accommodate sutures therethrough. In exemplary methods, the body 12 may be formed around the core wire by one or more of molding, casting, 3D printing, machining, and the like.

The outer material and resulting body 12 may have a substantially uniform cross-section around the perimeter of the body 12 around the central and end regions 14, 16, as shown, e.g., having a substantially circular, oval, or other cross-section, or, alternatively, the cross-section may be varied in different regions around the perimeter, if desired.

The rigidity of the device 10A may be substantially uniform around the perimeter or, alternatively, the rigidity may be varied around the perimeter of the body 12. For example, one of the end regions 16 that is intended to be positioned around the annulus adjacent a region of an anterior leaflet of the valve immediately adjacent the patient's aorta may be more flexible than the rest of the body 12, e.g., positioned around the annulus adjacent a posterior leaflet of the valve.

Turning to FIG. 1B, another device 10B is shown that includes a “C” shaped body 12, which may be constructed generally similar to the device 10A, e.g., including end regions 16 extending from opposite ends of a central or intermediate region 14. However, the device 10B also includes a flexible portion 18 extending between the ends of the “C” shaped body opposite the central region 14 to provide an enclosed ring. As shown in FIG. 1B, the flexible portion 18 may have a shape similar to the central region 16, e.g., a curved shape defining a radius of curvature larger than the end regions 16. Alternatively, the flexible portion 18 may be substantially straight and/or curved inwardly towards the central region 16, e.g., at the midpoint of the flexible portion 18, such that the body 12 defines a generally “D” shape.

The flexible portion 18 may be constructed of relatively soft and flexible material compared to the other regions of the body 12. For example, if the central and end regions 14, 16 of the body 12 include a core wire and polymeric material formed around the core wire, the flexible portion may be formed from pliable polymeric material without a core wire to increase its relative flexibility.

The central and end regions 14, 16 of the body 12 may lie substantially within a plane for positioning around the valve annulus, i.e., adjacent the anterior and posterior leaflets, and the flexible portion 18 may have sufficient flexibility to conform to a shape of the annulus adjacent the septal leaflet. For example, the flexible portion 18 may be sufficiently flexible to deflect out of the plane when placed against the valve annulus adjacent the septal leaflet to conform easily the native anatomical shape.

Optionally, the flexible portion 18 may include one or more demarcations, colors, or other indicators (not shown), e.g., on the fabric covering the flexible portion 18, to provide visual confirmation to the surgeon to avoid placing sutures through the flexible portion 18. For example, the fabric covering the flexible portion 18 that is intended to overly sensitive tissue structures adjacent the valve annulus, e.g., the bundle of His and the like (not shown), may include a solid color, a colored pattern, and the like, to provide a visual indication to the surgeon to avoid placing sutures or other fasteners through the identified region.

Turning to FIG. 2A, another example of an annuloplasty device 110 is shown that includes an enclosed ring or body 112 constructed generally similar to the other devices herein, e.g., including a core wire, polymeric material surrounding the core wire, and/or a fabric covering (not shown). FIGS. 2B-2E show additional views of the device 10C to show the shape of the device 110 around its perimeter.

As shown, the device 110 includes a body 112 including a central or intermediate region 114 and curved first and second end regions 116 extending from opposite ends of the central region 114, e.g., generally similar to the other devices herein. For example, the central region 114 may extend generally along a length of the body 112 and the end regions 116 may define opposite ends of the length of the body 112.

In addition, the device 10C includes a bridge 118 extending between the first and second end regions 116 opposite the central region 114 to define an enclosed ring. The bridge 118 may be configured to avoid interference with a septal leaflet of the tricuspid valve and/or conform to other anatomy of the patient, e.g., the septal wall and/or aorta of the patient's heart.

For example, as best seen in FIGS. 2C and 2D, the central and end regions 114, 116 generally lie within a plane 113 and the bridge 118 may extend out of the plane 113, e.g., to define an arch shape that extends transversely out of the plane 113. Optionally, the end regions 116 may deviate slightly from the plane 113, e.g., at the transition to the bridge 118, as shown, e.g., to conform to the shape of the native valve annulus. As viewed from above the plane, e.g., as shown in FIG. 2B, the central and end region 114, 116 may have curved shapes extending outwardly from the center of the body 112, and the bridge 118 may have a substantially straight shape or may have a curved shape, e.g., extending inwardly towards the center of the body 112 at its midpoint, similar to the device 10B.

However, as viewed along the plane, e.g., as best seen in FIG. 2C, the bridge 118 may define an arch shape with a midpoint 118a defining a maximum height “h” out of the plane 113 defined by the central and end regions 114, 116. Optionally, as shown in FIG. 2D, the arch shape of the bridge 118 may lie generally within a bridge plane 119 that intersects the plane 113 of the central and end regions 114, 116, e.g., defining an angle θ. The angle θ may be less than ninety degrees) (90°, such that the midpoint 118a of the bridge 118 is closer to the center of the body 112 than the regions that connect to the end regions 116. The angle of the bridge 118 may accommodate positioning the device 110 adjacent a tricuspid valve annulus 90 with the bridge 118 conforming to the adjacent septal wall and/or aorta, as described further elsewhere herein.

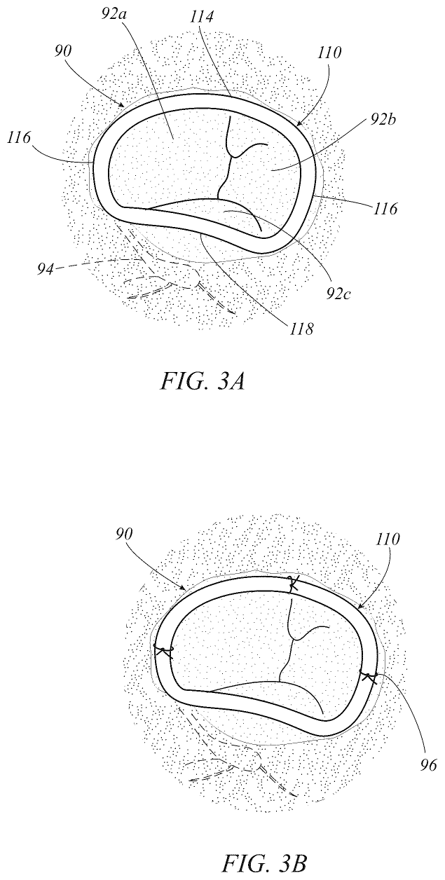

The rigidity of the device 110 may be substantially uniform around the perimeter, e.g., throughout the central and end regions 114, 116 and the bridge 118 or, alternatively, the rigidity may be varied around the perimeter of the body 112. For example, at least a portion of the first end region 116a, e.g., at a region 116c closest to the bridge 118 identified in FIG. 2B, may be more flexible than the central region 114 and the second end region 116b, e.g., to avoid interference with an aorta adjacent the tricuspid valve when the device 110 is implanted around the valve annulus 90, e.g., as shown in FIGS. 3A and 3B and described further elsewhere herein.

In one example, the body 112 may include a substantially rigid core wire (not shown) extending from the first end region 116a through the central region 114 to the second end region 116b. Optionally, the core wire may also extend from the first end region 116a through the bridge 118 to the second end region 116b, e.g., such that the core wire is an enclosed ring. Material may be provided around the core wire, e.g., rigid, semi-rigid, or flexible outer material, for example, elastomeric material such as silicone, a polymer such as urethane methacrylate, and the like.

The outer material and resulting body 112 may have a substantially uniform cross-section around the perimeter of the body 112 around the central and end regions 114, 116 and bridge 118, as shown, e.g., having a substantially circular, oval, or other cross-section. Alternatively, the cross-section may be varied in different regions around the perimeter of the body 112, if desired.

Fabric, e.g., Dacron and the like (not shown), may be provided around the body 112 to cover the outer surface and provide the finished device 110. The resulting device 110 may be substantially rigid or semi-rigid, e.g., to support the annulus 90 of the tricuspid valve, e.g., as shown in FIGS. 3A and 3B. Alternatively, the body 112 may be malleable, e.g., such that the shape may be modified by a surgeon to conform to the anatomy of an individual patient before implantation. In addition or alternative, multiple devices 110 may be provided having different sizes and shapes such that a device may be selected to correspond to the particular anatomy of an individual patient's valve annulus.

With additional reference to FIGS. 3A and 3B, the devices herein may be implanted around a tricuspid valve annulus 90, e.g., in a manner that avoid interference with a septal leaflet 92c of the valve and/or avoids risks of damaging conductive tissue and/or the bundle of His 94 underlying or otherwise adjacent the valve annulus 90, as shown in FIG. 3A. The tricuspid valve may be accessed using conventional open, minimally invasive, or transcatheter procedures.

For example, with respect to the device 10A shown in FIG. 1A, once the valve is exposed, the device 10A may be positioned around the valve annulus 90 (such as that shown in FIG. 3A) such that the tips 17 of the first and second end regions 16 are located on either side of a septal leaflet 92c of the tricuspid valve with a gap between the tips 17, and then the device 10A may be secured to tissue adjacent the annulus 90. For example, one or more sutures or other fasteners may be introduced through fabric covering the device 10A into the tissue adjacent the annulus.

The distance of the gap between the tips 17 of the first and second ends 16 may be sized to avoid placement of the tips 17 over the septal leaflet 92c when the device 10A is secured, e.g., by suturing the device 10A around the anterior and posterior leaflets 92a, 92b of the tricuspid valve. Similarly, the device 10B may be positioned around the valve annulus 90 such that the flexible portion 18 overlies the annulus adjacent the septal leaflet 92c. Sutures may be used to secure the device 10B with no sutures delivered adjacent the septal leaflet 92c, i.e., through the flexible portion 18, to avoid damaging conductive tissue or the bundle of His 94.

For the device 110 shown in FIGS. 2A-2E, turning to FIGS. 3A and 3B, the device 110 may be positioned around the tricuspid valve annulus 90 such that the central region 114 and first and second end regions 116 extend around the anterior and posterior leaflets 92a, 92b with the ends of the end regions 116 located on either side of a septal leaflet 92 of the tricuspid valve such that the bridge 118 overlies the septal leaflet 92c, as best seen in FIG. 3A. The bridge 118 may be positioned against or spaced apart from the septal wall (not shown) adjacent the valve annulus 90.

As shown in FIG. 3B, the device 110 may be secured to tissue adjacent the annulus 90, e.g., by introducing one or more sutures 96 (and/or other fasteners, not shown) through fabric covering the device 110 into the tissue adjacent the annulus 90, e.g., around the central and end regions 114, 116 without delivering the sutures 96 through the bridge 118 and any tissue adjacent the bridge.

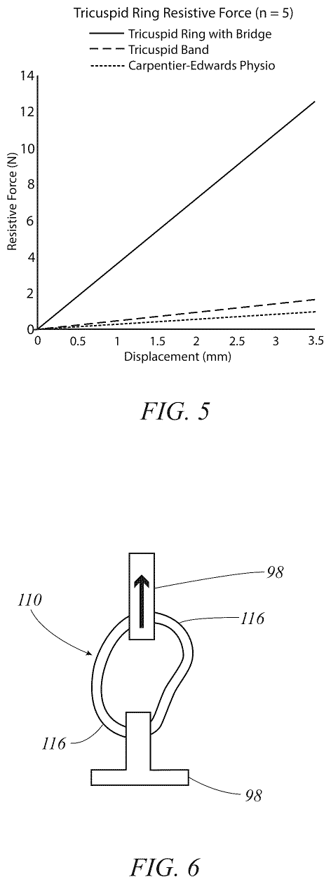

FIG. 4 shows an example of a test apparatus, e.g., an Instron 5848 Microtester, that was used to test resistive forces of annuloplasty devices, such as those described elsewhere herein. In the example shown, a body 112 of an annuloplasty device, such as that shown in FIGS. 2A-2E, was secured to the test apparatus, e.g., by mounting opposite end regions 116 to mounts 98 on the test apparatus and the mounts 98 were directed away from one another apply tensile forces to the body 110. FIG. 5 shows exemplary resistive forces that were measured from tests using the apparatus. It was found that both the device 110 (tricuspid ring with contoured bridge) and devices 10A, 10B (tricuspid band with or without flexible region 18) had improved resistive forces compared to currently available Physio tricuspid bands. It was found that the device 110 had a substantially higher resistive force, because the bridge 118 allowed for retention of the two end regions 116 of the body 112, and this may translate to improved durability and decreased recurrence of tricuspid regurgitation in implanted devices.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. An annuloplasty device for implantation around a tricuspid valve of a heart, comprising:
a body including a central region, first and second end regions extending from opposite ends of the central region, and a bridge extending between the first and second end regions opposite the central region to define an enclosed ring configured to avoid interference with a septal leaflet of the tricuspid valve.

2. The device of claim 1, wherein the central and end regions generally lie within a plane and wherein the bridge extends out of the plane.

3. The device of claim 2, wherein the bridge has an arch shape that extends transversely out of the plane.

4. The device of claim 3, wherein the arch shape of the bridge lies generally within a bridge plane that intersects the plane of the central and end regions.

5. The device of claim 4, wherein the bridge plane defines an acute angle relative to the plane of the central and end regions such that a midpoint of the bridge is closer to the central region than regions that transition from the bridge to the end regions.

6. The device of claim 1, wherein at least a flexible portion of the first end region is more flexible than the central region and the second end region to avoid interference with an aorta adjacent the tricuspid valve

7. The device of claim 6, wherein the flexible portion is located at a transition from the first end region to the bridge.

8. The device of claim 1, wherein the central region and the first and second end regions comprise curved regions extending outwardly within the plane relative to a center of the body.

9. The device of claim 8, wherein a radius of curvature of the central region is larger than a radius of curvature of the first and second end regions.

10. The device of claim 8, wherein the body defines a generally “D” shape viewed from above the plane.

11. The device of claim 1, wherein the body comprises a substantially rigid core wire extending from the first end region through the central region to the second end region and material surrounding the core wire to define an outer surface of the body.

12. The device of claim 11, wherein the core wire extends from the first end region through the bridge to the second end region.

13. The device of claim 11, wherein the core wire is an enclosed ring.

14. The device of claim 11, wherein the core wire terminates in the first and second regions and does not extend into the bridge.

15. The device of claim 11, wherein the material comprises one or more of an elastomer and a polymer.

16. The device of claim 1, further comprising fabric covering the body.

17. The device of claim 1, wherein the body is substantially rigid around its perimeter.

18. The device of claim 1, wherein the body is malleable.

19. An annuloplasty device for implantation around a tricuspid valve of a heart, comprising:
a body including a curved central region and curved first and second end regions extending from opposite ends of the central region lying generally within a plane, and a bridge extending between the first and second end regions opposite the central region to define an enclosed ring, the bridge extending out of the plane between the first and second end regions to avoid interference with a septal leaflet of the tricuspid valve.

20-33. (canceled)

34. A method for performing annuloplasty, comprising:
providing an annuloplasty device comprising a body including a central region and first and second end regions extending from opposite ends of the central region such that tips of the end regions are spaced apart from one another, thereby generally defining a “C” shape;
positioning the device around a tricuspid valve annulus such that the tips of the first and second end regions are located on either side of a septal leaflet of the tricuspid valve with a gap between the tips; and
securing the device to tissue adjacent the annulus.

35-42. (canceled)
